(19) United States
(12) Patent Application Publication
(10) Pub. No.: US 2023/0320629 A1
(43) Pub. Date: Oct. 12, 2023

(54) Drug Delivery Cannula with Continuous Glucose Monitoring Capability

(71) Applicant: Pacific Diabetes Technologies, Inc., Portland, OR (US)

(72) Inventors: Thomas Seidl, Portland, OR (US); William Kenneth Ward, Portland, OR (US); Florian Guillot, Portland, OR (US); Brennen McCullough, Portland, OR (US); Chad Knutsen, Portland, OR (US); Robert S Cargill, Portland, OR (US); Scott M. Vanderwerf, Portland, OR (US); Matt Breen, Portland, OR (US)

(73) Assignee: Pacific Diabetes Technologies, Inc., Portland, OR (US)

(21) Appl. No.: 18/132,589

(22) Filed: Apr. 10, 2023

Related U.S. Application Data
(63) Continuation of application No. PCT/US21/54956, filed on Oct. 14, 2021.
(60) Provisional application No. 63/091,729, filed on Oct. 14, 2020.

(51) Int. Cl. A61B 5/145; A61B 5/00; A61B 5/1486

(52) U.S. Cl. CPC A61B 5/14532; A61B 5/4839; A61B 5/6848; A61B 5/14865; A61M 2005/1726

(57) ABSTRACT

By combining analyte concentration monitoring electrodes and infusion functions into a single subcutaneous element, the described sensing cannulae having a rectangular cross-section avoids the need for two separate devices for insulin delivery and glucose concentration determination. The substantially flat, thus planar, surface of the sensing cannula provides a substrate for deposition of one or more sensing electrodes, preferably through a lithographic-type process. The inner lumen of the sensing cannula serves as a conduit for drug delivery and is formed in a manner that is compatible with lithographic-type electrode formation. The rectangular cross-section of the sensing cannula also allows face and side ports establishing fluid communication between the inner lumen and tissue that preferably reduces the incidence of occlusion of the inner lumen of the sensing cannula.

### REFERENCE TO RELATED APPLICATIONS

This application is a continuation of International Application No. PCT/US2021/054956, filed Oct. 14, 2021, entitled “Drug Delivery Cannula with Continuous Glucose Monitoring Capability”, which claims the benefit of U.S. Provisional Application No. 63/091,729 entitled “Planar Drug Delivery Cannula with Continuous Glucose Monitoring Capability” filed Oct. 14, 2020, both of which are incorporated by reference in the entirety.

### BACKGROUND

Many people with type 1 diabetes (T1D) deliver their insulin using portable insulin pumps that deliver insulin or insulin analogs subcutaneously (SC). This process has been termed continuous subcutaneous insulin infusion (CSII). These devices are now being coupled with continuous glucose monitors (CGMs) in systems that utilize real-time glucose values to adjust insulin delivery to the patient. However, existing systems require the use of two separate percutaneous devices: both a CGM sensor and an insulin infusion cannula.

Currently available solutions use two separate elements, a CGM sensor and an infusion cannula, mounted on a single combined housing. However, such solutions require separate elements as the mechanical elements to support both the individual sensor and infusion tube must be provided. It also requires multiple insertion needles and multiple insertion sites, increasing user discomfort and risk of skin infection. Therefore, there is an unmet need for devices that combine both into a single subcutaneous element.

An initial prototype of a combined device was developed using a strategy of fabricating a glucose oxidase (GOx)-based amperometric glucose sensor disposed on the surface of a flexible polymeric strip. Then, this strip was laminated around the surface of a 25-gauge needle. This allowed production of an array of many sensors at the same time in parallel. It also afforded the benefit of handling a planar substrate during the various deposition steps including enzyme and membrane deposition. The arrays were subsequently individualized using laser micro-milling and laminated to the surface of a preformed stainless-steel needle using an adhesive. This enabled testing of the ability to sense glucose concentration in the subcutaneous interstitial fluid in the presence of local insulin delivery.

Using this prototype, our group determined that conventional first-generation glucose sensors, those that measure hydrogen peroxide generation by glucose oxidase via electrochemical means, suffer from loss of sensitivity in the extended presence of insulin. This is the case in continuous glucose monitoring wherein individual sensors are typically used for 7-14 days. We discovered that this was due to electropolymerization of phenolic preservatives in the insulin formulation on the surface of the working electrode. We determined that the sensitivity decay could be avoided using a device that employs a redox-mediated chemistry. Compared to a peroxide-generating sensing system, an osmium-based redox mediator system allows substantial lowering of the electrode bias voltage necessary to capture electrons from the enzyme to develop a glucose responsive current.

The resulting sensor arrangement is capable of measuring glucose continuously in the presence of insulin and its preservatives in vivo in swine, as published in Diabetes Technol Ther. 2017 April; 19(4):226-236. (An Amperometric Glucose Sensor Integrated into an Insulin Delivery Cannula: In Vitro and In Vivo Evaluation. Ward W K, Heinrich G, Breen M, Benware S, Vollum N, Morris K, Knutsen C, Kowalski J D, Campbell S, Biehler J, Vreeke M S, Vanderwerf S M, Castle J R, Cargill R S.) This work is the subject of U.S. Patent App. US20160354542A1, incorporated herein by reference. Results of swine testing of this device (FIG. 1) are the first published demonstration of a redox-mediated amperometric sensor measuring interstitial fluid glucose levels following delivery of an insulin bolus into the same site.

This device has now been tested in human feasibility studies with results that are similar to those in swine (FIG. 2, from Peter G. Jacobs, Nichole Tyler, Scott M. Vanderwerf, Clara Mosquera-Lopez, et al., Measuring glucose at the site of insulin delivery with a redox-mediated sensor, Biosensors and Bioelectronics, 2020, 112221, ISSN 0956-5663). Amperometric sensor currents (circular symbols) fell briefly immediately following administration of an insulin bolus, but recovered quickly. Following this test, the sensor continued to track blood glucose values closely (square symbols) as determined by whole blood glucose reference measurements taken using a YSI 2300 analyzer.

The temporary decline in current immediately after insulin delivery was of the same duration and magnitude as the decline after saline delivery, consistent with interstitial fluid dilution, not a result of electrochemical effects or insulin per se. A commercially marketed sensor was also placed at a site far removed from the insulin infusion, per the manufacturer's instructions, to act as a comparator.

Although this device offered a suitable demonstration platform for proof of concept of glucose sensing at the same site as insulin delivery, this prototype exceeds 0.75 mm diameter, substantially larger than presently marketed infusion sets. Conventional steel infusion sets have infusion needles with cross-sectional areas that are 60-90% smaller than this 23-gauge form factor. To shrink the device to a more viable form factor, considerable effort was expended to laminate sensors to smaller gauge needles, but the extremely small diameter of such needles rendered this approach infeasible from a manufacturing standpoint.

An attempt was made to dispense the sensor elements directly on a preformed cannula, but adequate repeatability and adhesion could not be achieved to yield a usable, safe product. To overcome these failure mechanisms, we invented methods for constructing a miniaturized planar, flexible drug delivery cannula that also measures glucose level, as presented below.

This novel cannula design also addresses common weaknesses in simple drug infusion cannulas. One noted author considers the cannula and its tubing (“insulin infusion set” or IIS) the “Achilles' heel” of CSII and notes that IIS problems are often one of the common reasons that people with type 1 diabetes discontinue CSII. Some of the more common IIS failures are catheter occlusions. (Heinemann L., Krinelke L.: Insulin infusion set: the Achilles heel of continuous subcutaneous insulin infusion. J Diabetes Sci Technol 2012; 6:954-964). In fact, a survey of insulin pump users found that 71% of these patients noted insulin cannula occlusions (Liebner T., Holl R., Heidtmann B, et al.: Insulin pump en katheter: komplikationen im Kindes-und Jugendalter. Diabetologie and Stoffwechsel 2011; 6:S5). In its teaching materials, an authoritative body of professional diabetes educators, the American Association of Diabetes Educators (AADE), states that occlusions are often caused by cannula kinks.

An occlusion of an insulin delivery cannula is a very serious event. Such an event leads to a state of elevated blood glucose, which can lead to a potentially fatal disorder, diabetic ketoacidosis (DKA). Ponder S W, Skyler J S, Kruger D F, et al. Unexplained hyperglycemia in continuous subcutaneous insulin infusion: Evaluation and treatment. Diabetes Educ 2008; 34:327-333.AND Pickup J C., Yemane N., Brackenridge A, et al.: Nonmetabolic complications of continuous subcutaneous insulin infusion: a patient survey. Diabetes Technol Ther 2014; 16:145-149.

One of the reasons that hyperglycemia due to IIS occlusion is so dangerous is that it is silent and unexpected. For example, if a person overeats carbohydrates, he/she anticipates and expects the subsequent hyperglycemia and can give a correction insulin bolus to bring the glucose level down. In contrast, when there is no obvious precipitating event, hyperglycemia may go unnoticed by the patient, especially if he/she is not using CGM. Furthermore, even when it is noticed, administration of a correction insulin bolus through the cannula will not correct the hyperglycemia due to the occlusion. Van Bon et al reported that approximately 60% of patients with type 1 diabetes using CSII experienced at least one episode of unexplained hyperglycemia during a 13-week study period (van Bon A C., Bode B W., Sert-Langeron C, et al.: Insulin glulisine compared to insulin aspart and to insulin lispro administered by continuous subcutaneous insulin infusion in patients with type 1 diabetes: a randomized controlled trial. Diabetes Technol Ther 2011; 13:607-614).

To further exacerbate the problem, it is well known that insulin occlusion alarms are notoriously poor in the early detection of occlusions. In fact, it may take many hours for the occlusion alarm to sound and thus, for the patient to realize that an occlusion has occurred (van Bon A C., Dragt D., deVries J H.: Significant time until catheter occlusion alerts in currently marketed insulin pumps at two basal rates. Diabetes Technol Ther 2012; 14:447-448).

FIG. 1A provides glucose-sensor cannula results obtained in anesthetized nondiabetic swine using a conventional hydrogen peroxide sensing electrode during lis-pro insulin delivery. A spurious response to the insulin formulation is observed.

FIG. 1B shows mean data for a sensor cannulae during lis-pro insulin delivery—no such spurious rise in current was noted in these devices. In both FIG. 1A and FIG. 1B, glucose was clamped at euglycemia until minute 200, at which time hyperglycemia was induced by dextrose infusion. The infusion was then stopped to monitor the fall of the sensor signal. The large arrow indicates the insulin bolus.

FIG. 2 illustrates results obtained from a subject having Type 1 diabetes using an integrated cannula sensing system having an osmium-based redox mediator, a CGM sensor lacking the ability to deliver drugs, and a YSI reference instrument. Vertical axes show glucose level in mg/dL. Solid circles show continuous glucose data from the integrated system. Open squares show continuous blood glucose data from a YSI reference instrument, and the dashed symbols show continuous glucose data from the commercially available CGM sensor.

The large arrow indicates the timing when insulin was subcutaneously delivered through the sensing cannula. Following this insulin delivery is a transient downward artifact attributed to dilution of the interstitial fluid. Similar experiments in which saline rather than insulin was delivered show an artifact of the same duration and magnitude—thus the transient downward artifact is not related to the insulin itself.

For all these reasons, there is an unmet need for improvements in drug delivery cannulae (such as those that deliver insulin) incorporating (1) continuous glucose monitoring in a manufacturable planar configuration, (2) resistance to cannula kinking, and (3) resistance to cannula occlusion.

The present invention avoids or ameliorates at least some of the disadvantages of conventional devices/methods.

### SUMMARY

In one aspect, the invention provides a sensing cannula for delivering a drug and determining glucose concentration when subcutaneously implanted, the sensing cannula including a proximal end and a distal end; a planar top face including an electrode; a planar bottom face; and a thickness between the planar top face and the planer bottom face including an inner lumen, where the planar top face, the planar bottom face, and the thickness in combination provide a rectangular cross-section to the sensing cannula, and where the inner lumen provides fluid communication from the proximal end to the distal end of the cannula.

In another aspect of the invention, there is a method of making multiple units of the cannula, the method including forming a sheet assembly array on a metal stencil, the metal stencil including slots cut through the metal stencil and alignment features, where the sheet assembly array is formed on the metal stencil by bonding polymer sheets comprising conductive layers, and where the metal stencil comprises multiple inner lumen formers tensioned by the alignment features in the metal stencil; and singularizing the sheet assembly array to form the multiple units of the cannula.

Other systems, methods, features and advantages of the invention will be, or will become, apparent to one with skill in the art upon examination of the following figures and detailed description. It is intended that all such additional systems, methods, features, and advantages be included within this description, be within the scope of the invention, and be protected by the claims that follow. The scope of the present invention is defined solely by the appended claims and is not affected by the statements within this summary.

### BRIEF DESCRIPTION OF THE FIGURES

The invention can be better understood with reference to the following drawings and description. The components in the figures are not necessarily to scale, emphasis instead being placed upon illustrating the principles of the invention.

FIG. 1A provides glucose-sensor cannula results obtained in anesthetized nondiabetic swine using a conventional hydrogen peroxide sensing electrode during lis-pro insulin delivery.

FIG. 1B shows mean data for redox sensor cannulae during lis-pro insulin delivery—no such spurious rise in current was noted in these devices.

FIG. 2 illustrates results obtained from a subject having Type 1 diabetes using a sensing cannula, a CGM sensor lacking the ability to deliver drugs, and a YSI reference instrument.

FIG. 3a represents a perspective view of a sensing cannula having proximal end, distal end, an inner lumen for subcutaneous delivery of a drug, and outer surfaces including a top face and a bottom face.

FIG. 3b represents a cross section of the sensing cannula showing the layers from which the sensing cannula is assembled.

FIG. 3c represents a cross section of the sensing cannula in which the hollow tube is eliminated, leaving the inner lumen formed out of the flexible elastomer.

FIG. 3d represents a cross section of the sensing cannula in which the flexible elastomer is eliminated.

FIG. 3e represents a cross section of the sensing cannula in which the flexible elastomer and the thermoset or high-melting thermoplastic are both eliminated.

FIG. 4 represents a perspective view of sensing cannula having both electrodes on the same face, showing proximal end; distal end; an inner lumen for subcutaneous delivery of a drug such as an insulin formulation; and a top surface containing one or more working electrodes used for the purpose of amperometric analyte sensing, and one or more pseudo-reference electrodes.

FIG. 5a represents a perspective view of sensing cannula, showing that the cannula includes one or more ports distributed along one or both faces of the sensing cannula that permit fluid to leave the cannula at points other than distal tip.

FIG. 5b represents a perspective view of the sensing cannula, showing that the sensing cannula includes one or more of the ports distributed along the edge of one or both faces of the sensing cannula that permit fluid to leave the sensing cannula at points other than distal tip.

FIG. 6 represents a perspective view of a sensing cannula further equipped with an insertion device or trocar.

FIG. 7a represents a sensing cannula that avoids the need for a trocar to insert the sensing cannula into the skin.

FIG. 7b depicts distal tip of sensing cannula.

FIG. 8a represents a sheet assembly array of multiple sensing cannulas in a single sheet assembly.

FIG. 8b represents sensing cannula parts being welded together with a thermal bonding process to form sensing cannula.

FIG. 9a represents a stencil fabricated from flat metal shim stock including an assembly of undivided sensing cannula.

FIG. 9b represents a modified sensor pattern allowing for extension of the conductors, permitting the contacts to diverge from the fluid path.

FIG. 10 provides measured blood glucose concentrations in relation to time for the sensing cannula, a conventional CGM only system, and a BGM device.

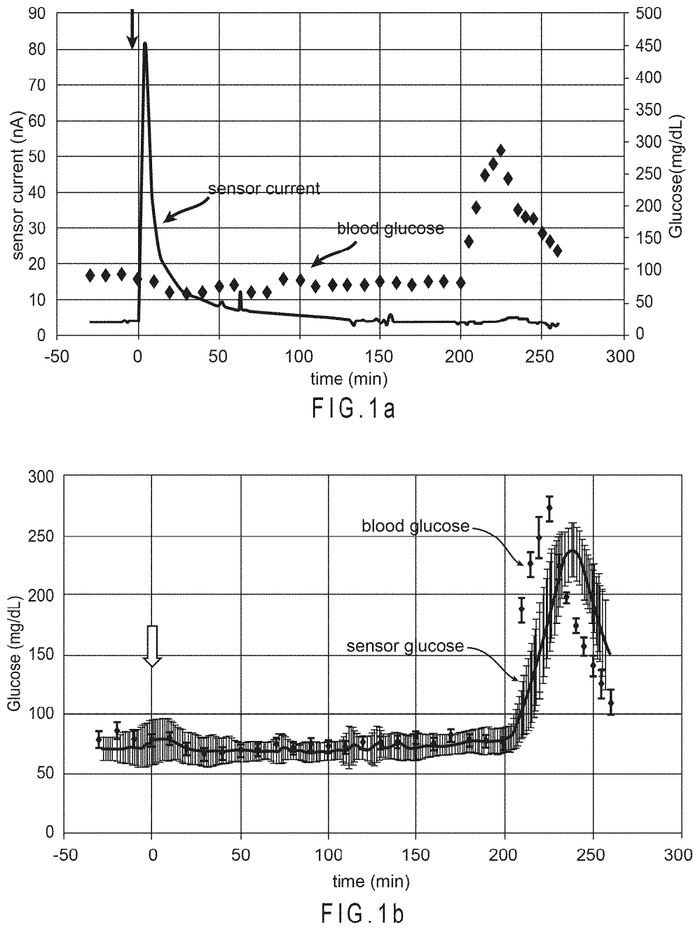

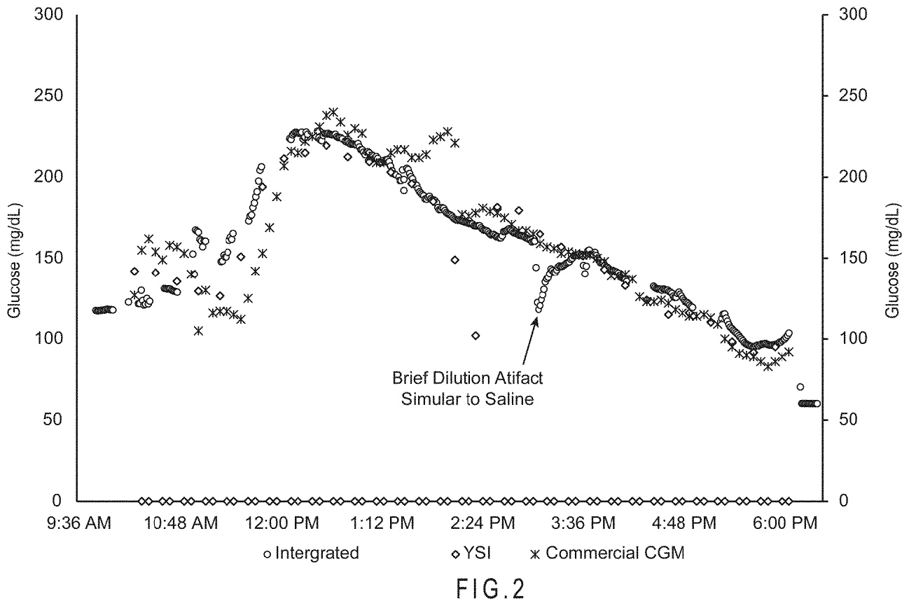

### DETAILED DESCRIPTION

By combining analyte concentration monitoring electrodes and infusion functions into a single subcutaneous element, the described sensing cannulae having a rectangular cross-section avoids the need for two separate devices for insulin delivery and glucose concentration determination. The substantially flat, thus planar, surface of the sensing cannula provides a substrate for deposition of one or more sensing electrodes, preferably through a lithographic-type process. The inner lumen of the sensing cannula serves as a conduit for drug delivery and is formed in a manner that is compatible with lithographic-type electrode formation. The rectangular cross-section of the sensing cannula also allows face and side ports establishing fluid communication between the inner lumen and tissue that preferably reduces the incidence of occlusion of the inner lumen of the sensing cannula.

The rectangular cross-section of the sensing cannula provides a thicker side-wall structure having greater resistance to occlusion due to movements and bending of the sensing cannula while subcutaneously inserted in the tissue of a subject in relation to conventional circular cannulae. Thus, the rectangular cross-section of the sensing cannula provides a safer solution to the user with less likelihood of a dangerous kink or occlusion that could interrupt drug delivery.

The inner lumen fluid path for drug delivery is formed within a sensing cannula substrate material during lamination of the sensing cannula substrate. This is possible because the polymer forming the inner lumen melts at a higher temperature than the glass transition temperature of the thermoplastic polymer surrounding the inner lumen. This construction avoids the challenge of laminating a completed sensor to an ever-smaller radius delivery needle. This construction also provides the benefit of manufacturing relatively large arrays of sensing cannulae in a sheet assembly using a stencil. When the individual sensing cannulae are separated from the sheet assembly, a sensing cannula having an inner lumen and an external rectangular cross-section is provided.

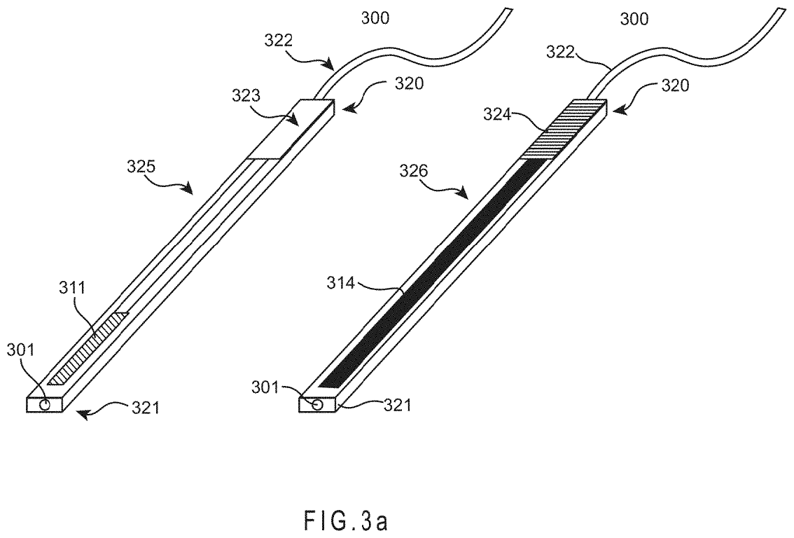

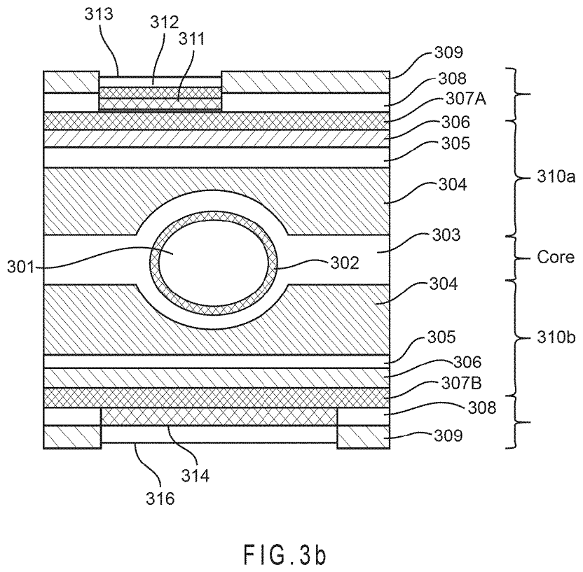

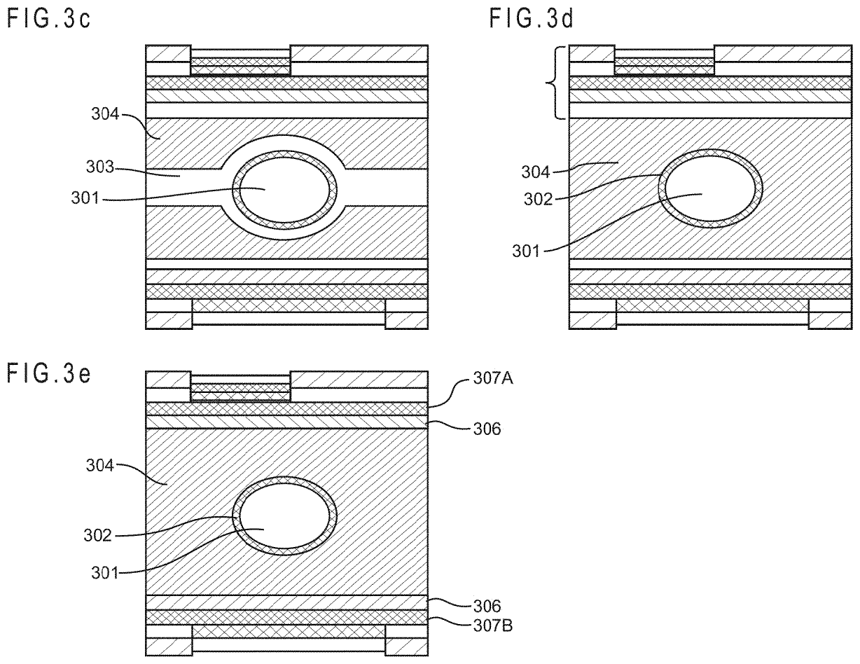

FIG. 3a represents a perspective view of a sensing cannula 300 having proximal end 320, distal end 321, an inner lumen 301 for subcutaneous delivery of a drug, and outer surfaces including a top face 325 and a bottom face 326. The sensing cannula 300 has a thickness or height between a planar top face 325 and a planar bottom face 326 of 0.1-0.6 mm, preferably 0.25-0.50 mm. The thickness provides a left-side wall and a right-side wall to the sensing cannula 300. The width of the sensing cannula 300 is typically 0.2-1.0 mm, preferably 0.3-0.7 mm. Preferably, the length of the sensing cannula 300 is 7-30 times the width, thus from 4-20 mm, preferably from 6-12 mm in length.

The substantially flat top face 325 and the substantially flat bottom face 326 of the sensing cannula 300 are amenable to the deposition of metal to form electrodes. The top face 325 includes one or more working electrodes 311 used for the purpose of amperometric analyte sensing, and the bottom face 326 may additionally contain a pseudo-reference electrode 314 or separate counter and reference electrodes (not shown).

During use, the distal end 321 of the sensing cannula 300 is typically immersed in subcutaneous interstitial fluid, with the sensing cannula 300 inserted through the skin of a mammal to permit subcutaneous drug delivery and analyte analysis. The proximal end 320 of the sensing cannula 300 extends out of the skin surface for electrical communication with the monitoring electronics of a measurement device, with the inner lumen 301 of the sensing cannula 300 in fluid communication with a source of the drug being delivered, such as a drug delivery pump, syringe, or gravity-fed source.

Electrical contact to biasing and monitoring circuitry is made through one or more contacts 323 at the proximal end 320 of the sensing cannula 300, and one or more contacts 324 if a pseudo-reference or counter and reference electrodes are present. In this embodiment, fluid is delivered into the sensing cannula 300 via a flexible polymeric tube 322 which may be composed of polytetrafluoroethylene (PTFE), polyurethane, polyolefin, polyimide, polyether ether ketone, silicone, or other material compatible with the drug in the given application and suitable for conveying a fluid in indirect communication with blood.

FIG. 3b represents a cross section of the sensing cannula 300 showing the layers from which the sensing cannula 300 is assembled. The sensing cannula 300 is formed from a flexible elastomer 303, such as polyurethane, surrounding a hollow tube 302 having the inner lumen 301 and an outer circumference. The hollow tube 302 may be metal, such as medical grade stainless steel, or polymeric, such as PTFE, polyimide, polyurethane, polyether ether ketone, polyethylene, or polypropylene. On the top and bottom faces of the flexible elastomer 303 are laminates 310a and 310b, each contacting a conductive layer. On both outer faces of the sensing cannula 300, adhesive layer 308 is adjacent to the outermost layer of polyimide 309.

The laminates 310a and 310b include a polymer bilayer comprising a low-melting point thermoplastic 304 such as polyethylene on the inner surface, a higher melting thermoplastic or thermoset layer 305 such as polyester in the middle, and an outer conductor 307a or 307b, which may be bonded to the polymer bilayer by way of an adhesive 306, or directly formed on the layer 305. The conductive layers may be composed of carbon paste or a metal foil such as copper, gold, silver, platinum, titanium, tantalum, niobium, or other conductive material, and serve to carry the electrical current generated by the electrode.

Attached to the upper conductor layer (outer conductor 307a as represented in the figure) are one or more working electrodes 311, including a thin film of gold, platinum, silver, or carbon. There is at least one working electrode having an enzyme and redox mediator layer 312. This layer typically contains glucose oxidase as a redox mediator and a cross-linking agent such as glutaraldehyde. Preferably, the enzyme and redox mediator layer 312 includes an osmium-based redox mediator having a ligand such as 4,4′-dimethyl-2,2′-bipyridine. The enzyme, redox mediator, or both may be bound to a pyridine- or imidazole-based electrically conductive polymer such as either poly (4-vinyl pyridine) or poly (1-vinyl imidazole) that serves to transfer electrons from the enzyme or redox mediator to the electrode surface.

The enzyme may be an engineered form of glucose oxidase or glucose dehydrogenase that permits direct coupling of electrons from the enzyme to the electrode surface. The enzyme and redox mediator layer 312 preferably is covered in a thin permselective membrane 313 that regulates the diffusion of the analyte and oxygen. This membrane may be comprised of a polymer such as poly vinyl pyridine, polyurethane, sulfonated tetrafluoroethylene-based fluoropolymers, such as NAFION™, or another suitable polymer.

In this embodiment, pseudo-reference electrode 314 serves as a combined counter and reference electrode and is located on the opposite face of the sensing cannula 300. The pseudo-reference electrode 314 is typically composed of silver/silver chloride or platinum covered by a membrane 316. The working electrode 311 and the pseudo-reference electrode 314 are connected through their respective conductor layers to a pair of contacts 323 and 324 at the proximal end of the sensor. The working electrode 311 typically has a surface area that is not more than 20-25% of the area of the pseudo-reference electrode 314.

For enhanced stability, the working, counter, pseudo-reference, or reference electrodes may be applied directly to a metal foil which can be composed of titanium or other element such as gold, niobium, or tantalum. Although metal foils can be electrodeposited or sputtered, there are distinct differences between these deposited foils and foils that have been made through mechanical means, as the porosity and grain structure of a rolled foil give it added resistance to fatigue and to moisture penetration. For this reason, a rolled metal foil is generally superior for fatigue resistance and adhesion. The metal foil can be bonded to the underlying base polymer using an adhesive material such as an epoxy, acrylate, or polyurethane.

People of ordinary skill in the art are familiar with many alternative conductive materials including, but not limited to, thin films, such as sputtered or evaporated metals, graphene, and conductive polymers, such as polyaniline. It should also be noted that there are processes by which metal foil can be bonded to a base polymer by adhesive-less methods.

There are many methods by which the working and pseudo-reference electrodes can be applied to the conductor layer. These methods include metal sputtering, electrodeposition, application of a metal ink, and metal evaporation through a mask. Typically, the working electrode is made from a film of gold or carbon, for example in a thickness of 30-900 nm, but preferably 30-200 nm. Typically, the pseudo-reference electrode is made from a film of silver, for example in a thickness of 200-4000 nm, but preferably 500-1500 nm. After the silver portion of the pseudo-reference electrode is deposited, silver chloride can be generated electrolytically by application of a current in an electrolyte medium that includes KCl and/or HCl. Alternatively, silver chloride can be generated chemically by exposure to aqueous ferric chloride or applied as an ink or paste. The pseudo-reference electrode may also be comprised of a metal foil, typically 2-12 μm in thickness, or possibly as thick as 50-100 μm.

FIG. 3c represents a cross section of the sensing cannula 300 in which the hollow tube is eliminated, leaving the inner lumen 301 formed out of the flexible elastomer 303.

FIG. 3d represents a cross section of the sensing cannula 300 in which the flexible elastomer 303 is eliminated. The low-melting thermoplastic 304 of the conductor layers contacts the hollow tube 302 directly.

FIG. 3e represents a cross section of the sensing cannula 300 in which the flexible elastomer 303 and the thermoset or high-melting thermoplastic 305 are both eliminated. The core of the sensing cannula 300 is formed by thermoplastic 304 bonded to conductors 307a and 307b.

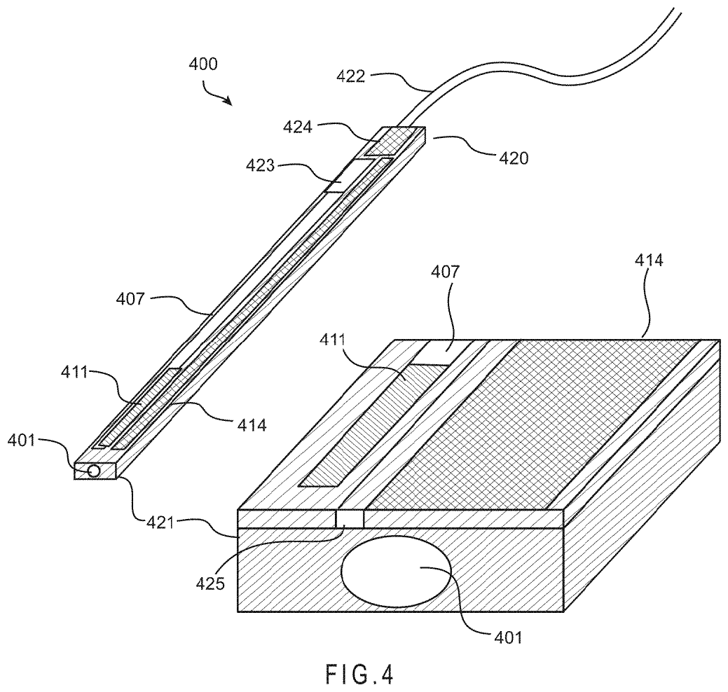

Electrode surfaces may be included on opposite faces of the sensing cannula 300 as in FIG. 3a, or with all electrodes on a single face as shown in FIG. 4. Both a working electrode and a pseudo-reference electrode can be disposed on one face of the sensing cannula 300 if the proper electrical separation is maintained. It should be noted that the conductive material underlying the working and pseudo-reference electrodes cannot be continuous, or else it would cause an electrical short between the two electrodes.

For this reason, the two conductors are separated by an insulating element. One method of separating the conductor into two parts is to use standard photolithography techniques including the use of sulfuric acid to etch away a channel in a metal foil such as titanium. Another is to ablate the metal channel using laser micromachining.

FIG. 4 represents a perspective view of sensing cannula 400 having both electrodes on the same face, showing proximal end 420; distal end 421; an inner lumen 401 for subcutaneous delivery of a drug such as an insulin formulation; and a top surface containing one or more working electrodes 411 used for the purpose of amperometric analyte sensing, and one or more pseudo-reference electrodes 414.

Electrical contact to biasing and monitoring circuitry for the one or more working electrodes is made through one or more contacts 423 at the proximal end of the sensing cannula 400. Electrical contact to biasing and monitoring circuitry for the one or more pseudo-reference electrode is made through one or more broadened contacts 424 at the proximal end of the sensing cannula 400.

If located adjacent to one another, working electrode 411 and pseudo-reference electrode 414 are preferably separated by an insulating strip 425. The working electrode 411 includes unchloridized region 407 and the region on which the enzyme and the redox mediator are deposited 411. Most or all the chloridized portion of the working electrode 411 is configured to reside subcutaneously. Unchloridized region 407 is configured to be used as a conductor and thus no chloridization is needed.

Fluid may be delivered into the sensing cannula 400 via a flexible polymeric tube 422 which may be composed of PTFE, polyurethane, polyethylene, polyimide, polyether ether ketone, silicone, or other material compatible with the drug in the given application and suitable for conveying a fluid in indirect communication with blood. The flexible polymeric tube 422 is in fluid communication with a source of a liquid drug, such as a drug delivery pump.

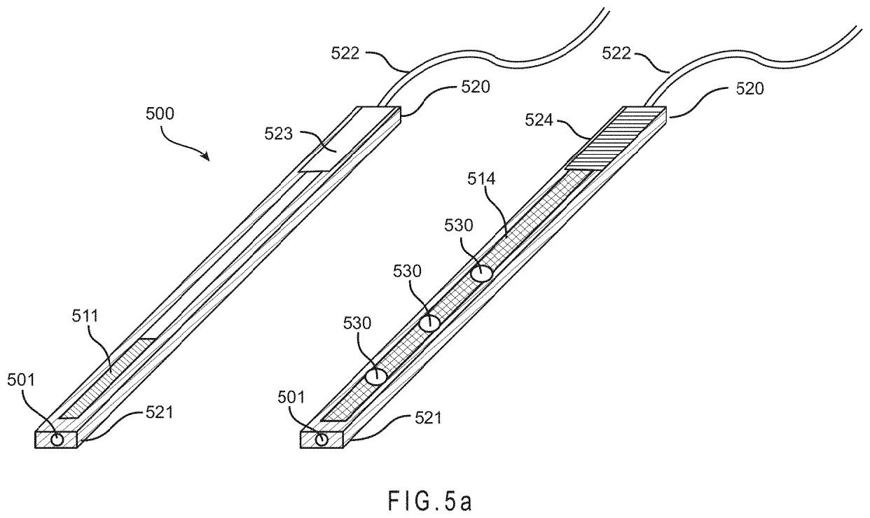

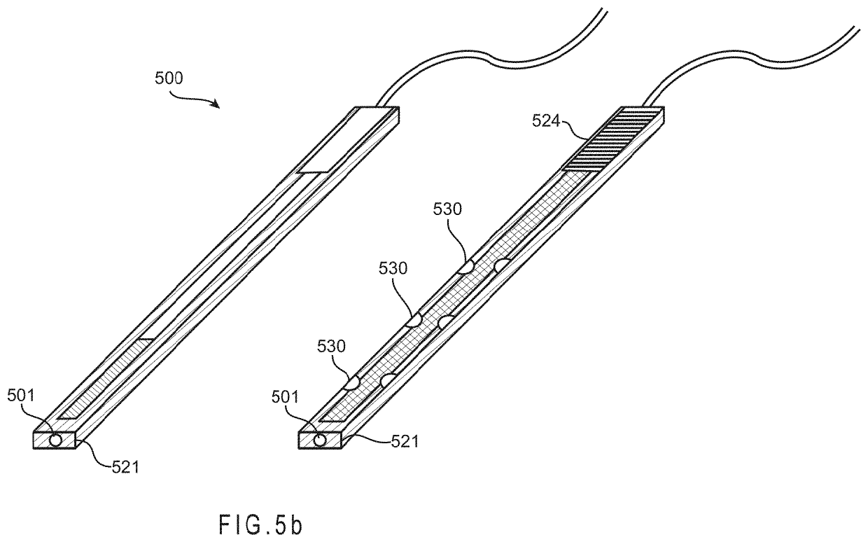

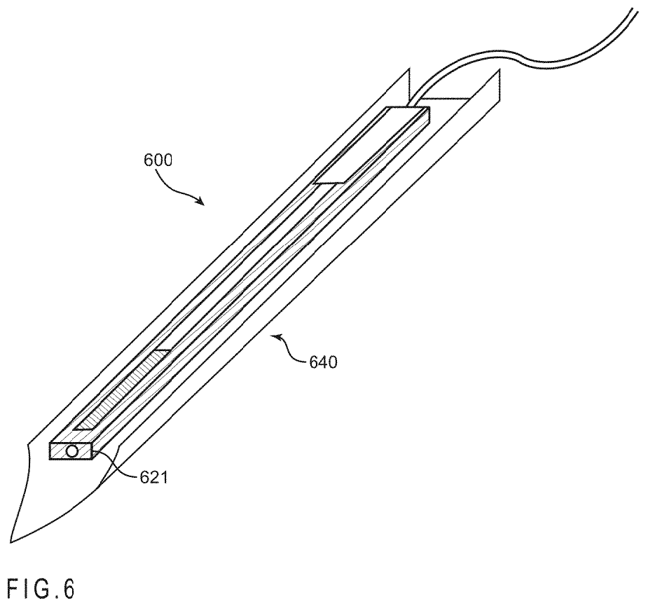

FIG. 5a represents a perspective view of sensing cannula 500, showing that the cannula includes one or more ports 530 distributed along one or both faces of the sensing cannula 500 that permit fluid to leave the cannula at points other than distal tip 521. This allows redundancy in case of obstructions and moves the insulin infusion to sites other than the focal point of the trauma from insertion. The distal tip of the sensing cannula 500 may be hollow, or it may be completely solid, thus lacking inner lumen 501, to increase the mechanical strength of the sensing cannula 500.

FIG. 5b represents a perspective view of the sensing cannula 500, showing that the sensing cannula 500 includes one or more of the ports 530 distributed along the edge of one or both faces of the sensing cannula 500 that permit fluid to leave the sensing cannula 500 at points other than distal tip 521. This allows redundancy in case of obstructions and moves the insulin infusion to sites other than the distal tip 521 which is the focal point of the trauma from insertion. In these embodiments, the distal tip 521 of the sensing cannula 500 may be hollow, or it may be completely solid, thus lacking inner lumen 501, to increase the mechanical strength of the sensing cannula 500.

FIG. 6 represents a perspective view of a sensing cannula 600 further equipped with an insertion device or trocar 640. The trocar 640 is used to pierce the skin and deliver distal end 621 of the cannula into the subcutaneous tissue, with typically 6-18 mm of the sensing cannula 600 below the skin surface. The insertion of the sensing cannula 600 into the skin may be perpendicular to the skin surface, or at an angle of 30-45 degrees from the plane of the skin. The trocar 640 is removed shortly after the sensing cannula 600 is placed properly under the skin.

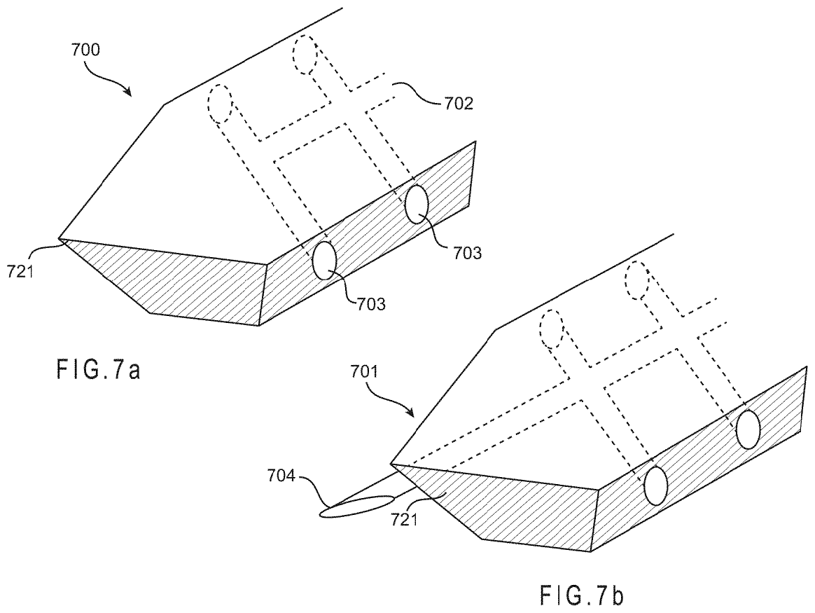

FIG. 7a represents a sensing cannula 700 that avoids the need for a trocar to insert the sensing cannula 700 into the skin. FIG. 7a depicts a distal tip 721 of sensing cannula 700, showing that the leading edge of the sensing cannula 700 has been tapered to a point to facilitate insertion of the sensing cannula 700 through the skin.

Different tip configurations (e.g., a three-sided point, different bevel angles) can be created to reduce pain and minimize deflection of the tip during insertion into tissue. The sensing cannula 700 is also shown with one or more lateral ports 703 connecting to at least one inner lumen 702 that permit fluid to leave the cannula at points other than the distal tip 721. This allows redundancy in case of obstruction and moves the drug infusion to sites other than the distal tip 721, which is the focal point for the trauma from insertion. The lateral ports can be created in the sensing cannula 700 by mechanical means (such as the presence of pins or similar inclusions temporarily present during the pressing/laminating process) or can be created by laser ablation, mechanical drilling, and the like.

FIG. 7b depicts distal tip 721 of sensing cannula 701. The leading edge of the sensing cannula 701 is tapered to a point to facilitate insertion through the skin. The sensing cannula 701 is also shown with one or more lateral ports 703 that permit fluid to leave the cannula at points other than the distal tip 721.

The sensing cannula 701 is shown with a metal tube 704, such as a stainless-steel tube, typically having an inner diameter of 100-300 um and an outer diameter of 125-400 um. In the sensing cannula 701, the metal tube 704 extends beyond the distal tip 721 of the elastomeric core for the ease of insertion.

The metal tube 704 can be left in place or, to minimize pain, removed after insertion. Another advantage of the metal tube 704 is to provide additional stiffness to the sensing cannula 701, thus minimizing the tendency for the sensing cannula 701 to buckle during insertion. In another embodiment, insertion of the sensing cannula 701 is eased by including a stiff reinforcing rod or rods in the polymer core (not shown).

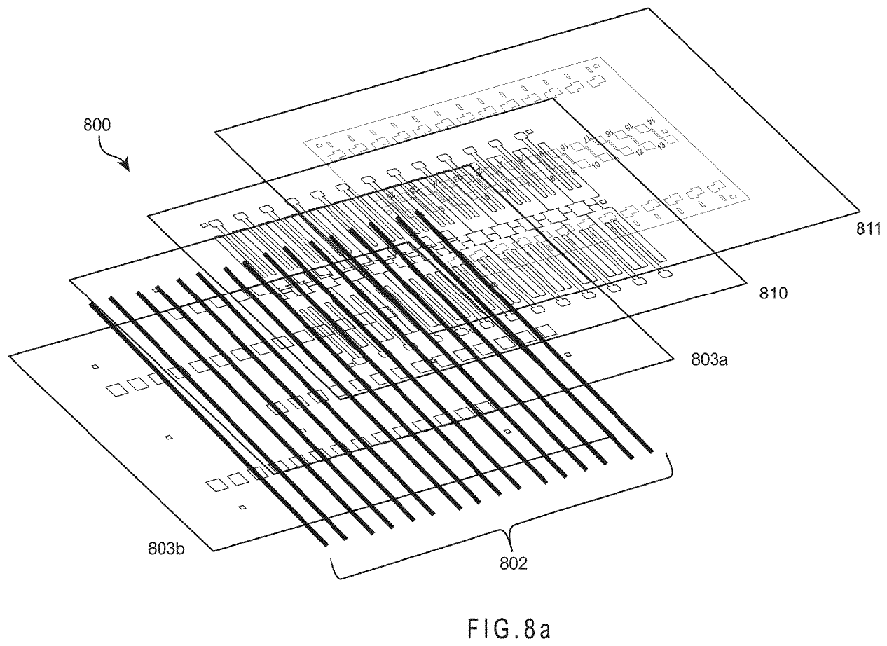

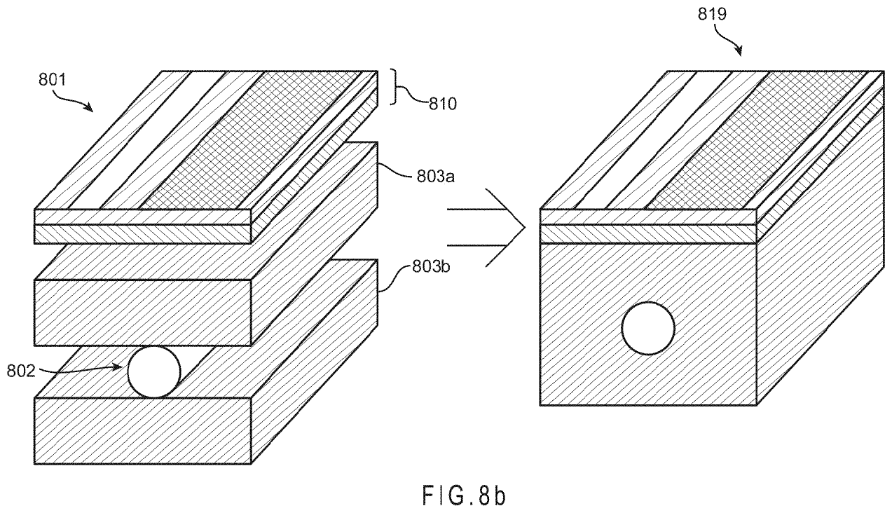

FIG. 8a represents a sheet assembly array 800 of multiple sensing cannulas in a single sheet assembly. The manufacturing process preferably creates a sheet assembly of sensing cannulae rather than a single cannula. The multiple sensing cannula elements are bonded to one another with direct thermal bonding. Polymer tubes 802 for inner lumen formation are placed between sheets forming the polymer core 803a, 803b. The conductive layer(s) 810 are placed against the outside surface of the polymer core and the inside surface of cover sheet 811. Alternatively, the polymer core can be omitted; in such a case, the conductive layer (or other conductive material) is bonded directly to the polymer tubes 802.

The polymer core layers 803a, 803b can be made from the same or different materials. This entire stack of materials is placed between the metal platens of a press and heated above the glass transition temperature (tg) of the polymer(s) comprising the core. As pressure is applied at this temperature, the core layers 803a, 803b soften or melt to change shape so that they flow around the polymer tube 802 forming the inner lumen.

The temperature at which the polymer tube 802 softens or melts preferably is chosen to be greater than the glass transition temperature (tg) of polymer core 803a, 803b. The greater tg of the polymer tube 802 in relation to the tg of the polymer core 803a, 803b results in the polymer tube 802 retaining its circular or elliptical shape after the polymer core 803a, 803b materials cool to room temperature and harden.

FIG. 8b represents sensing cannula parts 802, 803a, 803b, and 810 being welded together with a thermal bonding process to form sensing cannula 819. The cover sheet 811 is omitted from the representation. The seams between the layers will not delaminate as the thermal welding process exceeds the tg of the polymer core 803a, 803b materials. Methods other than a heated press can be used to join the polymer material, including ultrasonic welding, laser welding, other types of polymer welding or other methods that can join two polymeric surfaces.

There are many possible polymers that can serve as the materials forming the polymer core 803a, 803b, including, but not limited to, poly-ether imide (PEI), polyethylene, polypropylene, polyvinyl chloride, polystyrene, polybenzimidazole, acrylic, nylon, and fluoropolymers such as polytetrafluoroethylene and others, all of which are thermoplastic polymers. Such materials become soft and flexible at certain temperatures and solidify on cooling. One example of appropriate materials would be PEI for the polymer core 803a, 803b and polyimide for the polymer tube 802. The glass transition temperature of PEI is 217° C.

The maximum service temperature for polyimide and two other heat-resistant polymers are shown in Table 1 as follows:

TABLE 1

| | Maximum Service | Melting |
|---|---|---|
| Polymer Type | Temperature | Point |
| Polyimide (e.g., VESPEL ®) | 300° C. | None |
| (Polyamide-imide) TORLON ® | 260° C. | None |
| Polyphenylene sulfide (RYTON ®) | 218° C. | None |

Table 1 demonstrates that both polyimide and polyamide-imide are suitable materials to be used for the polymer tube 802 in the scenario in which PEI is used for the polymer core 803a, 803b. The polymer tube 802 is preferably composed of thermoplastic polymer whose tg is greater than the tg of the polymer core 803a, 803b, or it can be a thermoset material such as polyimide, epoxy, urea formaldehyde, phenolics, unsaturated polyester resins, or others. In either instance, the material forming the polymer tube 802 retains its shape at the temperature at which the polymer core 803a, 803b bonds to the polymer tube 802 and the other layers of the sensing cannula 801.

It can be desirable to avoid the need for an external metal trocar during the insertion process. Such rigid trocars tend to add to the pain experienced by the patient during insertion. Therefore, in some embodiments, it may be desirable for the sensing cannula 801 to have added stiffness, such as the situation in which the sensing cannula 801 is inserted into the subcutaneous tissue without use of an external metal tube or partial tube known as a trocar.

In this instance, the materials forming the polymer core 803a, 803b can be stiffened by the addition of fibers including, but not limited to, carbon fiber or glass fiber. When the material from which the sensing cannula 801 is formed retains its formed shape, the polymer tube 802 cross-sectional area can be expanded without compromising the stiffness of the sensing cannula 801. In such a case, the inner lumen formed by the polymer tube 802 can constitute up to 50-90% of the total cross-sectional cannula area. Another method of stiffening the sensing cannula 801 is to include metal wires or a metallic mesh within the polymer tube 802 during the lamination process. Such metal reinforcing material could be used as an electrical conductor, a mechanical stiffener, or both.

Referring to FIG. 8b, the sensing cannula 801 is sufficiently thick to avoid insulin delivery stoppage (occlusion due to kinking and the like) even when a large bending force is applied to the sensing cannula 801, such as when an applied force bends the proximal and distal ends of the sensing cannula towards each other until they are parallel.

While not shown in the figure, there can be an additional fluid passage tube or tubes, oriented parallel to tube 802, such as the situation in which greater flow or fluid path redundancy through the sensing cannula 801 is desired.

The polymer part of the sensing cannula, excluding the metal electrode part, can be one polymer material, e.g., a polymer matrix with a tubular opening passing through it. Such a sensing cannula can be manufactured by an extrusion procedure in which release-coated wires are present initially during the extrusion and later removed. Alternatively, a polymer tube constructed of the same material as the polymer matrix is present during the extrusion process (as the tube is surrounded by polymer) and left in place.

Description of the Stencil

One method of producing a sensing cannula in a planar shape consistent with the described embodiments involves laminating the layers of the sensing cannula around an internal stencil. The stencil serves to define individual sensing cannulae. It also aids in separating the sensing cannulae from each other after manufacture, as the stencil provides a clearer edge while retaining the ability to process singulated sensors in a single planar array.

Laser singulation (separation of the manufactured sensing cannulae) is one method for separating a single sensing cannula from manufactured sheet assemblies of multiple sensing cannula. However, thermoplastic materials that are relatively low melting, like polyethylene (PE), are known to be difficult to laser cut, and we have observed that the heat affected zones of the lasering process suffer degradation of the mechanical structure of the sensing cannulae. When cutting a relatively small part like the electrodes, the proportion of the part that is exposed to heat is increased and deleterious effects are more noticeable. The effect on electrode performance is a degradation of mechanical structure resulting in lowered electrode durability when the electrode is exposed to the bending and shear forces during normal use. The lowered durability increases the chance of electrode breakage and loss of signal when subcutaneously inserted.

Use of a stencil mitigates the effect of excess heating and embrittlement along the laser cut line (heat affected zone) by reducing the amount of material that needs to be cut through. During the array lamination process, the steel stencil replaces the core layer in the negative space of the electrode pattern, leaving only the conductive layer to cut through to obtain a singulated sensing cannula. The stencil reduces the amount of material to cut during singulation by occupying those areas of an array core layer that normally would be “offcuts”. With less material to cut through the laser power may be decreased and/or the cut speed increased.

The stencil improves the singulation process and is also expected to be critical in conferring the alignment needed for achieving the precise pitch needed for a scalable glucose sensor production process. The alignment enables automated enzyme coating and other downstream processes that rely on alignment. Finally, the stencil further permits the use of softer core layers that would not be possible to detach using conventional methods, for example, thermoplastic polyurethane (TPU) or a soft poly-(dimethyl siloxane) or PDMS. Importantly, the stencil allows all the above to be carried out with control over the shape of the resulting singulated parts.

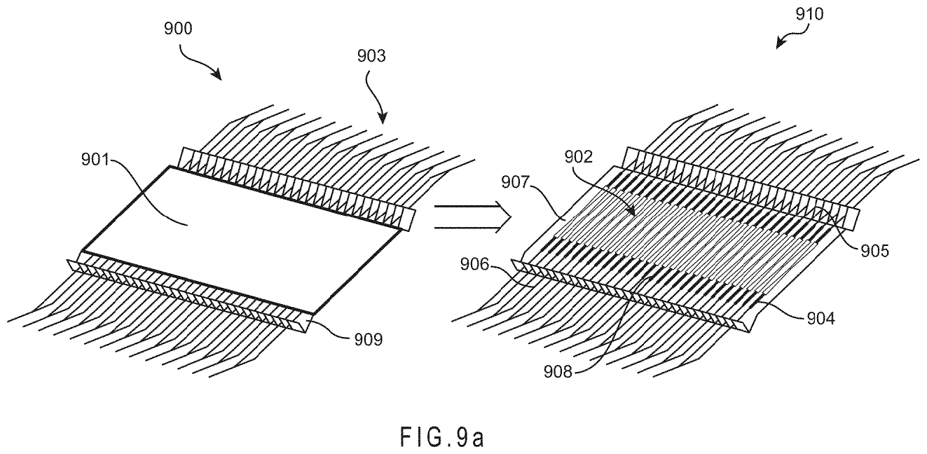

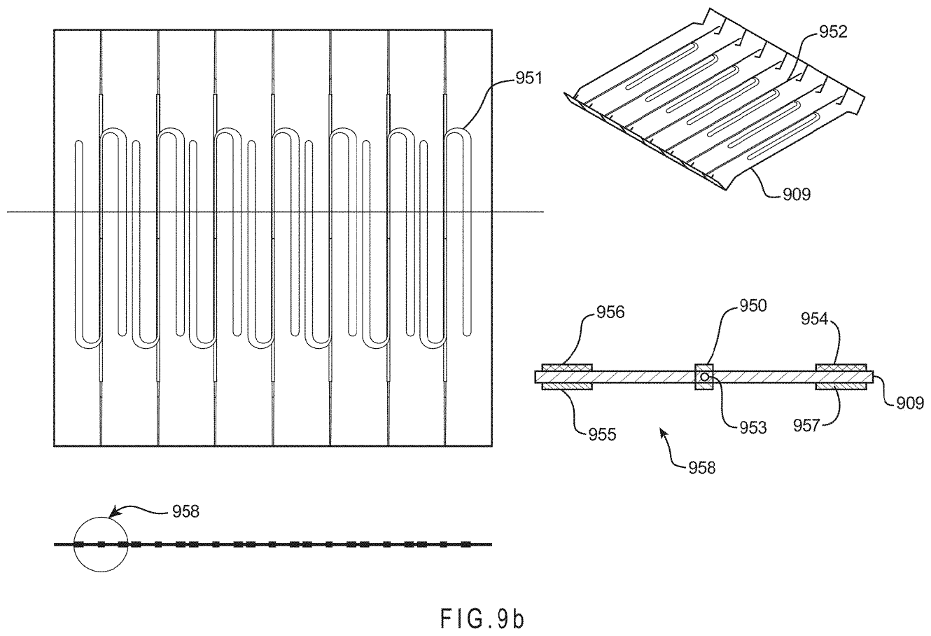

FIG. 9a represents an assembly stencil 900 including metal stencil 909, an assembly of undivided sensing cannulae 901, and tubes or mandrel 903 that will form the inner lumen of the sensing cannula when divided. Thus, in addition to a polymer tube a solid metal stabilizing rod or mandrel may serve as an inner lumen former when the polymer tube is omitted or used in conjunction with the polymer tube during manufacture to assist in aligning the polymer tube. Also not shown in the figure are the slots (negative regions) cut in the metal stencil 909 in the shape of the sides of the sensing cannula. Thus, a significant portion of the thickness of the sensing cannulae resides within the height or thickness of the metal stencil 909.

The metal stencil 909 preferably is fabricated from flat metal shim stock (4-20 mils, typically 12 mils), but may be fabricated from other materials compatible with the sensing cannula manufacturing process. The conductive layers (not shown) of the undivided sensing cannulae 901 form continuous films on the top and bottom of the metal stencil 909. The tubes or mandrel 903 are secured in the metal stencil 901 and tensioned during lamination of the sensing cannulae to maintain the position of the tubes or mandrel 903 within the sensing cannulae. Mandrels are used when the inner lumen of the sensing cannulae are not formed with the inclusion of a separate tube.

The shim stock forming the metal stencil 909 is preferably laser-cut to pattern into it a regular array of negative spaces with an outline matching that of the sensing cannulae. As such, the metal stencil 909 makes up the negative spaces of the sensing cannula array. Aside from the electrode shaped pattern, key design features include: 1) a continuous metal frame that confers mechanical stability to the metal stencil 909 and 2) fine alignment notch features 904 are adjacent to the proximal end of each sensing cannula. The fine alignment notch features 904 serve to align the tubes or mandrel 903 that form the inner lumen of the sensing cannulae. The fine alignment notch features 904 preferably also are positioned within a bend that is introduced into the metal stencil 909 so that the frame of the metal stencil 909 is out of the way of and does not interfere with the tubes or mandrel 903 during lamination.

While the metal stencil 909 serves to align the tubes or mandrel 903, a lamination mold using a two-stage tensioning procedure is preferred to assist in ensuring that alignment is maintained throughout the sensing cannula array fabrication process. Rough alignment features 905 that are not continuous with the exterior pattern of the sensing cannulae are not essential and serve as a positioning aid while stringing the mold and tensioning the tubes or mandrel 903. The resulting partially cut array enables batch processing of sensing cannulae.

The areas of the metal stencil 909 corresponding to the positive space of the stencil may be used to separate the outermost layers from the core layer of the sensing cannulae upon singulation. This is relevant to mechanically decoupling the conductive layers from each other and from the fluid path.

When the assembly stencil 900 is singularized via laser to separate the sensing cannula and the offcut regions are removed, finished assembly stencil 910 results. Negative spaces in the metal stencil 909 occupy most of the space previously occupied by the assembly of undivided sensing cannulae 901. Fine alignment features 904 of the metal stencil 909 are sized to appropriately constrain tubes 903, thus providing a precise location for the inner lumen through the sensing cannulae. Rough alignment features 905 in the metal stencil 909 provide approximate positioning of the tubes or mandrel 903 prior to molding of the assembly of undivided sensing cannulae 901. When tubes are used, free ends 906 of the tubes may be cut to the desired length prior to removing the individual sensing cannula from the array. Two sensing cannulae are formed with connected distal ends sharing a single tube or mandrel 903. Thus, two sensing cannulae are mirror images of each other on either side of a center line of the metal stencil 909. Extra negative space 908 in the metal stencil 909 allows for excess thermoplastic to flow out during molding of the assembly of undivided sensing cannulae 901.

FIG. 9b represents front cross-section 958 of sensing cannula formed on and through metal stencil 909 where formed external conductor wires 951 are represented. Fluid path tubing 952 is shown with ends trimmed before the sensing cannula is removed from the metal stencil 909. The cross-section 930 of the metal stencil 909 shows the sensing cannula 950 with what will become an inner lumen. The external conductor wires 951 of the sensing cannula are laser cut on the surface of the metal stencil. Negative metal stencil region 953, thus a slot cut through the metal stencil 909, permits thermoplastic to flow in and around the tube 952, thus forming the inner lumen of the sensing cannula.

External conductor wires 954 are in electrical communication with the working electrode and external conductor wires 955 are in electrical communication with the pseudo-reference electrode of the represented sensing cannula. External conductor wires 956, 956 are in electrical communication with the working and pseudo-reference electrodes of the distal end attached sensing cannula that is not shown due to the cross-section.

The sensing cannula may be inserted in the subcutaneous tissue in a human either manually or using an automated insertion device. This sensing cannula serves the dual purposes of continuous glucose monitoring and insulin delivery. The tubing attached to the proximal end of the sensing cannula is typically attached to an insulin pump such as one manufactured by Medtronic, Animas, Nipro, Sooil, Tandem, SFC Fluidics or Insulet. Typically, a handheld controller allows the user to observe the current subcutaneous interstitial glucose levels, the recent and remote glucose concentration and concentration trends, and possibly also the insulin delivery rate (current, recent or remote). In addition, the sensing cannula can provide glucose data to an algorithm that controls the delivery of insulin (and optionally other drugs such as glucagon), to serve as an artificial endocrine pancreas or automated insulin delivery system.

Although certain embodiments have been illustrated and described herein, it will be appreciated by those of ordinary skill in the art that a wide variety of alternate and/or equivalent embodiments or implementations calculated to achieve the same purposes may be substituted for the embodiments shown and described without departing from the scope. Those with skill in the art will readily appreciate that embodiments may be implemented in a very wide variety of ways. This application is intended to cover any adaptations or variations of the embodiments discussed herein. Therefore, it is manifestly intended that embodiments be limited only by the claims and the equivalents thereof.

The following examples illustrate one or more preferred embodiments of the invention. Numerous variations may be made to the following examples that lie within the scope of the invention.

### EXAMPLES

### Example 1: Electrode Formation for a Sensing Cannula

The following chemical procedure in which chemicals are deposited to form the working electrode may be used to form the sensing function of the sensing cannula: A redox mediator (RM) such as osmium bound to dimethyl bipyridine was synthesized and purified. For additional details, see U.S. patent application Ser. No. 15/169,432 “Avoidance of insulin preservative-induced interference in biosensors.” The RM was then chemically combined with a polymer such as polyvinyl imidazole. The resulting compound is known as a redox mediator polymer (RMP). One benefit of including an RMP is that the polarizing bias required to poise an RMP-type sensor (often in the 0-200 mV range) is typically much lower than the bias needed to poise a non-RMP sensor. The value of the low bias potential is that it avoids oxidizing (and creating a glucose-like signal not responsive to sample glucose concentration) excipients commonly found in insulin formulations, such as phenol and meta-cresol. However, in the case in which an insulin formulation does not contain these phenolic preservatives, it may be possible to bias the sensor at a higher potential and thus eliminate the RMP material. For a discussion of the effect of insulin formulation preservatives on glucose sensor design, see publication: Diabetes Technol Ther. 2017 April; 19(4):226-236. An Amperometric Glucose Sensor Integrated into an Insulin Delivery Cannula: In Vitro and In Vivo Evaluation. Ward W K, Heinrich G, Breen M, Benware S, Vollum N, Morris K, Knutsen C, Kowalski J D, Campbell S, Biehler J, Vreeke M S, Vanderwerf S M, Castle J R, Cargill R S.

The RMP was combined with glucose oxidase and glutaraldehyde, for example, in a w/w/w ratio of 6:3:5 and immediately deposited on the working electrode by aerosol printing, dropcasting, ink jet printing, dip coating or some other controlled method of deposition. An outer, glucose-limiting membrane such as poly-4-vinyl pyridine co-styrene and/or a polyurethane was applied by any of the above deposition methods (typically applied to the working electrode and the pseudo-reference electrode). This membrane reduces glucose influx and provides a tough membrane to protect the sensor.

### Example 2: Continuous Glucose Monitoring with a Sensing Cannula

A sensing cannula and a non-drug delivery capable CGM system were used to determine blood glucose levels of a Type 1 diabetic individual over an approximately four-day period. Reference blood glucose readings were obtained with a blood glucose monitoring (BGM) system relying on finger sticks and disposable test sensors.

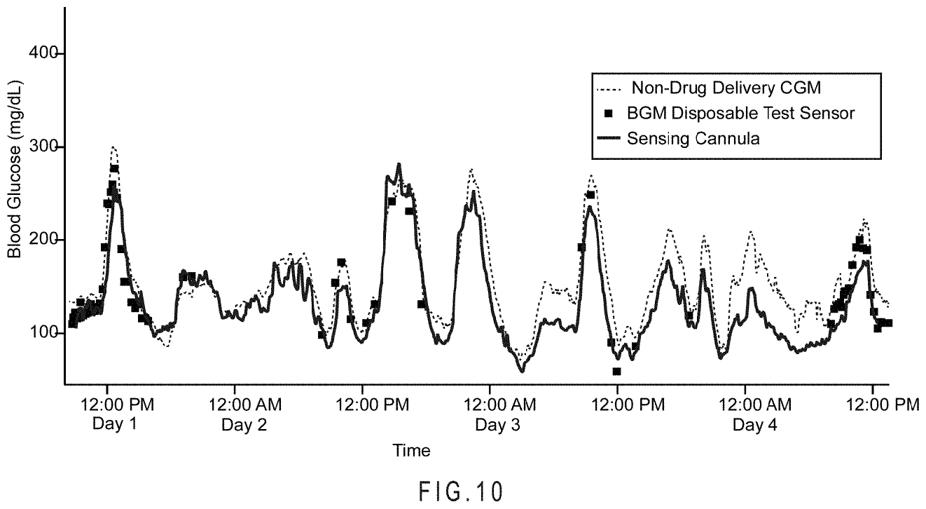

FIG. 10 provides measured blood glucose concentrations in relation to time for the sensing cannula, a conventional CGM only system, and a (BGM) device. As can be seen from the graph the sensing cannula provided accurate blood glucose readings in comparison to the BGM system. Throughout the analysis the MARD (the mean absolute relative difference, a representation of concentration sensing accuracy) was determined to be 11.3% for the sensing cannula versus 13.6% for the conventional CGM sensor lacking an inner lumen and thus lacking the ability to deliver a drug while monitoring sample glucose concentration.

To provide a clear and more consistent understanding of the specification and claims of this application, the following definitions are provided.

Cannula: a structure having a hollow core, an outer wall, and two ends, with one or more openings at each end.

Continuous Glucose Monitoring (CGM): the continuous or frequent sampling of interstitial or blood glucose levels by a sensor placed into a human or animal.

Continuous Subcutaneous Insulin Infusion (CSII): the provision of a continuous or frequent supply of insulin via a temporary or permanent cannula inserted through the skin of a human or animal.

Microneedle: a small needle or array of needles having dimensions generally less than 1 mm used to penetrate the stratum corneum and provide access to the dermis for transcutaneous delivery, sampling, or withdrawal of drugs or bodily fluids.

Stencil: an impervious material such as a sheet of foil cut with a pattern into which a substance such as a molten polymer or resin is forced to create a predefined three-dimensional shape

Metal Foil: a thin, flat piece of metal having little or no porosity and a density approximating that of the constituent elements. A metal foil is a flexible metal layer which is at least 2 micrometers in thickness and is typically no larger than 12 micrometers in thickness.

Pseudo-reference electrode: a combination counter/reference electrode. This type of combination electrode is possible when the reference electrode materials are separated, by their insolubility, from the reaction components of the analysis solution. Counter/reference electrodes are typically a mixture of silver (Ag) and silver chloride (AgCl), which exhibits stable electrochemical properties due to the insolubility of its components in the aqueous environment of the sample. If the ratio of Ag to AgCl is not significantly changed during use, the electrochemical properties of the electrode do not significantly change during use.

Singulation: the process of separating an array of sensing cannulae into discrete and individual sensing cannula.

Rectangular cross-section means the cross-section of the sensing cannula cut through a diameter of the inner lumen. Rectangular includes distorted rectangles and squares, thus parallelograms.

Various operations may be described as multiple discrete operations in turn, in a manner that may clarify embodiments; however, the order of description should not be construed to imply that these operations are order dependent.

The description may use perspective-based descriptions such as up/down, back/front, and top/bottom. Such descriptions are merely used to facilitate the discussion and are not intended to restrict the application of disclosed embodiments.

The terms “coupled” and “connected,” along with their derivatives, may be used. These terms are not intended as synonyms for each other. Rather, “connected” may be used to indicate that two or more elements are in direct physical or electrical contact with each other. “Coupled” may mean that two or more elements are in direct physical or electrical contact. However, “coupled” may also mean that two or more elements are not in direct contact with each other, but still cooperate or interact with each other.

For the purposes of the description, a phrase in the form “A/B” or in the form “A and/or B” means (A), (B), or (A and B). For the purposes of the description, a phrase in the form “at least one of A, B, and C” means (A), (B), (C), (A and B), (A and C), (B and C), or (A, B and C). For the purposes of the description, a phrase in the form “(A)B” means (B) or (AB) that is, A is an optional element.

The description may use the terms “embodiment” or “embodiments,” which may each refer to one or more of the same or different embodiments. Furthermore, the terms “comprising,” “including,” “having,” and the like, as used with respect to embodiments, are synonymous, and are generally intended as “open” terms (e.g., the term “including” should be interpreted as “including but not limited to,” the term “having” should be interpreted as “having at least,” the term “includes” should be interpreted as “includes but is not limited to,” etc.).

With respect to the use of any plural and/or singular terms herein, those having skill in the art can translate from the plural to the singular and/or from the singular to the plural as is appropriate to the context and/or application. The various singular/plural permutations may be expressly set forth herein for sake of clarity.

Unless otherwise indicated, all numbers expressing distances, quantities, and the like used in the specification and claims are to be understood as indicating both the exact values as shown and as being modified by the term “about”. Thus, unless indicated to the contrary, the numerical values of the specification and claims are approximations that may vary depending on the desired properties sought to be obtained and the margin of error in determining the values. At the very least, and not as an attempt to limit the application of the doctrine of equivalents to the scope of the claims, each numerical parameter should at least be construed considering the margin of error, the number of reported significant digits, and by applying ordinary rounding techniques.

Unless the context clearly dictates otherwise, where a range of values is provided, each intervening value to the tenth of the unit of the lower limit between the lower limit and the upper limit of the range is included in the range of values.

The terms “a”, “an”, and “the” used in the specification claims are to be construed to cover both the singular and the plural, unless otherwise indicated or contradicted by context. No language in the specification should be construed as indicating any non-claimed element to be essential to the practice of the invention.

Spatially relative terms, such as “top”, “bottom”, “right”, “left”, “beneath”, “below”, “lower”, “above”, “upper”, and the like, may be used for ease of description to describe one element or feature's relationship to another element(s) or feature(s) as illustrated in the figures. Spatially relative terms are intended to encompass different orientations of the device in use or operation in addition to the orientation depicted in the figures. For example, if the device in the figures is turned over or rotated, elements described as “below”, or “beneath” other elements or features would then be oriented “above” the other elements or features. Thus, the exemplary term “below” can encompass both an orientation of above and below. The device may be otherwise oriented (rotated 90 degrees or at other orientations) and the spatially relative descriptors used herein interpreted accordingly.

The simplified diagrams and drawings do not illustrate all the various connections and assemblies of the various components, however, those skilled in the art will understand how to implement such connections and assemblies, based on the illustrated components, figures, and provided descriptions.

While the present general inventive concept has been illustrated by description of several example embodiments, and while the illustrative embodiments have been described in detail, it is not the intention of the applicant to restrict or in any way limit the scope of the general inventive concept to such descriptions and illustrations. Instead, the descriptions, drawings, and claims herein are to be regarded as illustrative in nature, and not as restrictive, and additional embodiments will readily appear to those skilled in the art upon reading the above description and drawings. Additional modifications will readily appear to those skilled in the art. Accordingly, departures may be made from such details without departing from the spirit or scope of applicant's general inventive concept.

The term “subject” refers to an animal, including, but not limited to, a primate (e.g., human, monkey, chimpanzee, gorilla, and the like), rodents (e.g., rats, mice, gerbils, hamsters, ferrets, and the like), lagomorphs, swine (e.g., pig, miniature pig), equine, canine, feline, and the like.

“Electrical communication” includes at least one of electrically connected and non-electrically connected: where electrically connected means components communicate with each other by means of a conducting path such as through a wire, a cable, other conductors, circuitry, combinations, and the like; and non-electrically connected means components communicate with each other with or without a conducting path such as with radio signals, lasers, cellular or other telephones, WIFI (wireless fidelity) or other wireless network protocols, satellites, combinations, and the like. Components with electrical communication may be both electrically connected and non-electrically connected; for example, components may be electrically connected to supply electrical power and non-electrically connected to transfer data and operating signals. “Electrical communication” also includes when components are operatively connected to perform a particular function.

1. An A sensing cannula for delivering a drug and determining glucose concentration when subcutaneously implanted, the sensing cannula comprising:
a proximal end and a distal end;
a planar top face comprising an electrode;
a planar bottom face; and
a thickness between the planar top face and the planer bottom face comprising an inner lumen,
where the planar top face, the planar bottom face, and the thickness in combination provide a rectangular cross-section to the sensing cannula, and
where the inner lumen provides fluid communication from the proximal end to the distal end of the cannula.

2. The cannula of claim 1 where the electrode of the planar top face is a working electrode and the planar bottom face comprises a pseudo-reference electrode.

3. The cannula of claim 1 where the electrode of the planar top face is a working electrode and the planar bottom face comprises a counter electrode and a reference electrode.

4. The cannula of claim 1 where the electrode of the planar top face comprises a working electrode and a pseudo-reference electrode.

5. The cannula of claim 1 where the electrode of the planar top face comprises a working electrode, a counter electrode, and a reference electrode.

6. The cannula of claim 1, where a width of the planar top face and the planar bottom face is from 0.2 mm to 1.0 mm.

7. The cannula of claim 6 where a length of the cannula defined by the inner lumen is seven to thirty times the width.

8. The cannula of claim 1 where the inner lumen is in fluid communication with a source of a drug, the source of the drug chosen from a drug delivery pump, a syringe, and a gravity-fed source.

9. The cannula of claim 1 where a distal end of the inner lumen constitutes from 50% to 90% of a cross-sectional area of the distal end of the cannula.

10. The cannula of claim 1 where the inner lumen is formed from a polymeric tube.

11. The cannula of claim 10 where the polymeric tube comprises a polymer chosen from polytetrafluoroethylene, polyurethane, polyolefin, polyimide, polyether ether ketone, silicone, epoxy, urea formaldehyde, phenolics, unsaturated polyester resins, and combinations thereof.

12. The cannula of claim 10 where the polymeric tube has a melting temperature that is higher than the glass transition temperature of surrounding polymer, where the surrounding polymer contacts the polymeric tube and contributes to the thickness between the planar top face and the planer bottom face of the cannula.

13. The cannula of claim 12 where the surrounding polymer is a thermoplastic polymer chosen from poly-ether imide, polyethylene, polypropylene, polyvinyl chloride, polystyrene, polybenzimidazole, acrylic, nylon, fluoropolymers, and combinations thereof.

14. The cannula of claim 12 where the surrounding polymer comprises fibers chosen from carbon fiber, glass fiber, and combinations thereof.

15. The cannula of claim 1 further comprising an electrical contact on the planar top face at the proximal end, where the electrical contact is in electrical communication with the electrode.

16. The cannula of claim 1 further comprising a thin permselective membrane on the electrode, where the thin permselective membrane contacts an enzyme and a redox mediator.

17. The cannula of claim 1 where the electrode has a surface area that is not more than 20% to 25% of the surface area of a pseudo-reference electrode.

18. The cannula of claim 1 further comprising at least one port establishing fluid communication between the inner lumen and at least one of the planar top face, the planar bottom face, and the thickness.

19. The cannula of claim 18 where the inner lumen lacks fluid communication with the distal end.

20. The cannula of claim 1 further comprising a trocar contacting at least one of the planar top face and the planar bottom face.

21. The cannula of claim 1 where the distal end is tapered to a point.

22. The cannula of claim 1 where a distal end of the inner lumen is in fluid communication with a metal tube extending beyond the distal end of the cannula.

23. A method of making multiple planar top and planar bottom face sensing cannula from a sheet assembly array, the method comprising:
forming a sheet assembly array on a metal stencil, the metal stencil including slots cut through the metal stencil and alignment features,
where the sheet assembly array is formed on the metal stencil by bonding polymer sheets comprising conductive layers, and
where the metal stencil comprises multiple inner lumen formers tensioned by the alignment features in the metal stencil; and
singularizing the sheet assembly array to form the multiple planar top and planar bottom face sensing cannula.

24.-35. (canceled)
